(19) United States
(12) Patent Application Publication
(10) Pub. No.: US 2014/0302999 A1
(43) Pub. Date: Oct. 9, 2014

(54) METHOD FOR DETERMINATION OF PHARMACOLOGICAL PROPERTIES OF RECOMBINANT PROTEINS

(71) Applicant: Magdalena Leszczyniecka, Cambridge, MA (US)

(72) Inventor: Magdalena Leszczyniecka, Cambridge, MA (US)

(21) Appl. No.: 14/355,390

(22) PCT Filed: Nov. 2, 2012
(86) PCT No.: PCT/US2012/063279

§ 371 Date: Apr. 30, 2014

Related U.S. Application Data
(60) Provisional application No. 61/555,206, filed on Nov. 3, 2011.

(51) Int. Cl. G01N 33/557

(52) U.S. Cl. CPC G01N 33/557
USPC 506/1; 506/9; 506/18

(57) ABSTRACT

The present invention is directed to a method for obtaining an in vitro pharmacological model of a recombinant protein drug in a given host. A plurality of biomolecules are selected which are known or suspected to influence pharmacology of the recombinant protein in the host via a binding interaction with the recombinant protein. The recombinant protein is contacted with each selected biomolecule and the binding kinetics parameters for each interaction are determined using a binding assay. These steps are then repeated with all selected biomolecules to produce a plurality of binding kinetics parameters for the selected biomolecules. The combined results provide an in vitro pharmacological model of the recombinant protein in the host. The in vitro pharmacological model may then be used in several applications, such as optimizing new batches of recombinant protein drugs, developing biosimilar or bio-better drug candidates.

### FIELD OF THE INVENTION

The present invention is in the technical field of analytical testing of recombinant proteins and more particularly, in vitro modeling of pharmacological profiles of post-translationally modified recombinant proteins.

### BACKGROUND OF THE INVENTION

Recombinant proteins are produced in living cells and represent the major class of biologic drugs used to treat a wide range of diseases. Examples of cells which are commonly used to produce recombinant proteins as active drug ingredients include mammalian cells such as Chinese Hamster Ovary cells (CHO), murine myeloma NSO cells, Baby Hamster Kidney (BHK) cells, or bacteria such as E. coli. Mammalian cells can modify recombinant proteins by adding a variety of post-translational modifications, such as glycosylation, carboxylation, hydroxylation, sulfation and amidation, among others.

Glycosylation refers to attachment of oligosaccharides to proteins and represents the most commonly found post-translational modification of recombinant proteins. Oligosaccharides consist of monosaccharide units that are connected to each other via glycosidic bonds. Oligosaccharides may also be branched, with each of the sugar units in the saccharine serving as an optional branching point. The oligosaccharide chains are attached to proteins co-translationally or post-translationally, via specific asparagine (N-linked) or serine/threonine (O-linked) residues. For N-linked glycosylation the consensus amino acid sequence of recombinant protein is Asn-X-Ser/Thr. O-Sulfation entails the attachment of a sulphate group to tyrosine, serine and threonine residues mediated by sulfotransferases Amidation is characterized by the replacement of the C-terminal carboxyl group of a protein with an amide group. γ-carboxylation and β-hydroxylation modifications are mediated by specific carboxylase and hydroxylase enzymes, with conversion of target glutamate residues to γ-carboxyglutamate (Glu→Gla) and either target conversion of aspartate residues to β-hydroxyaspartate (Asp→Hya) or asparagine residues to β-hydroxyasparagine (Asn→Hyn).

During the manufacture of recombinant proteins, different batches of the same drug are frequently modified by structurally distinct post-translational modifications, some of which may affect half-life, biodistribution, clearance, therapeutic effectiveness and safety of the drug. Because of the need to monitor this variability, a wide variety of methods have been developed to analyze the structural diversity of post-translational profiles of recombinant proteins. These methods include: chromatography, capillary electrophoresis, mass spectrometry, and plant lectin arrays.

Unfortunately, structural analysis of the quantity and type of post-translational modifications is not sufficient to establish the safety and efficacy profile of a new batch of a recombinant protein because the complexity of the biological interactions prevents simple correlations of structure-activity relationships of the drug and the host. Currently, in vivo animal models are used to obtain information about the impact of changes in post-translational modifications on the pharmacological profile. These studies are very laborious and expensive and not conducive to testing a large number of samples.

Pharmacological properties of recombinant protein drugs are controlled by a series of interactions between the recombinant protein and the host proteins following drug administration. Post-translational modifications of recombinant proteins are likely to interact with host proteins present in the bloodstream and surrounding tissues. Since different batches of a recombinant protein are modified by different post-translational modifications, they are likely to have different pharmacological profiles as they interact with different host proteins.

Some of the examples of host proteins likely to interact with recombinant proteins include but are not limited to lectins. Lectins belong to a diverse family of structurally unrelated proteins classified under one name because of their glycan binding properties, each with different sugar specificity and different localization. The extracellular subgroup of lectins is most likely to interact with recombinant protein drugs as the members of this group are either secreted into the extracellular matrix or body fluids, or localized on or within the plasma membrane where they mediate a range of functions including cell adhesion, cell signaling, glycoprotein clearance and pathogen recognition. This lectin class includes C-type lectins, R-type lectins, siglecs, ficolins, 1-type lectins and galectins. Recombinant protein drugs in the immunoglobulin G class (IgG) are also known to interact with host FeRn receptor. FcRn functions as a “salvage receptor” which regulates levels of circulating IgGs.

### SUMMARY OF THE INVENTION

The present invention provides methods and systems for pharmacological characterization of recombinant proteins.

Since structural analysis of post-translational modifications cannot predict the pharmacological profile of a given recombinant protein in vivo and testing of a recombinant protein manufactured under a multitude of growth conditions cannot be achieved in animals (due to a large number of samples), in vitro methods that model the in vivo pharmacological profiles of recombinant protein drugs would provide a significant advancement in the field of analytical testing.

It has been recognized that current analytical methods are incapable of predicting the impact of structural changes to post-translational profiles on the pharmacological properties of the recombinant protein drugs. Methods for assessing these effects will be increasingly needed as generic copies of original biological drugs (also known as “biogenerics,” “biosimilars” and “follow-on biologics”) are being developed for the western markets. Methods and systems are also needed to rapidly assess the impact of variations in production conditions on the pharmacological profiles of recombinant protein drugs. These methods and systems are furthermore expected to provide a means for analyzing changes in the pharmacological profiles of existing recombinant protein used as drugs, and improving their pharmacological properties thereby leading to production of so-called “bio-better” drugs.

In one aspect of the invention, there is provided a method for obtaining an in vitro pharmacological model for a recombinant protein drug in a host. The method includes the steps of:

a) selecting a plurality of biomolecules which are known or suspected to influence the pharmacological profile of the recombinant protein in a host via one or more binding interactions with the recombinant protein;

b) contacting the recombinant protein with one of the biomolecules;

c) determining binding kinetics parameters of the recombinant protein to the biomolecule using a binding assay; and

d) repeating steps b) and c) with additional biomolecules from the group selected above to produce a set of binding kinetics parameters for the different biomolecules. The set of binding kinetics parameters provides an in vitro pharmacological model of the recombinant protein.

The binding interactions may represent interactions of the biomolecules with the backbone of the recombinant protein or may represent interactions with a post-translational modification of the recombinant protein. The post-translational modifications may include, but are not limited to any of the following post-translational modifications: glycosylation, carboxylation, hydroxylation, O-sulfation, amidation, glycylation, glycation, alkylation, acylation, acetylation, phosphorylation, biotinylation, formylation, lipidation, iodination, prenylation, oxidation, palmitoylation, pegylation, phosphatidylinositolation, phosphopantetheinylation, sialylation, and selenoylation.

The recombinant protein being analyzed may be a monoclonal antibody, a fusion protein, a Fab fragment, or any other recombinant protein suitable as a biological drug candidate.

The method described above may be used for assessment of a new batch, lot or a biosimilar of the recombinant protein of interest produced under different production conditions or at different locations and their comparison to the reference product. An example of a reference product is a recombinant protein in a formulation (or isolated from a formulation) of an approved and branded pharmaceutical product. The reference product concept will also be understood by a person skilled in the art to encompass a recombinant protein which is not an approved and branded pharmaceutical product, but instead a copy of the active ingredient of such a branded product which is understood to have desirable pharmacological properties. To facilitate the analysis, the in vitro pharmacological model for a reference product is produced under identical conditions as those used for obtaining the in vitro pharmacological model of a new batch, lot or a biosimilar of the recombinant protein of interest. Comparison of the in vitro pharmacological model of a new batch, lot or a biosimilar of the recombinant protein of interest with the in vitro pharmacological model for a reference product provides an assessment of the pharmacological profile of the altered version of the recombinant protein of interest with respect to the reference product.

Additional steps may be then taken which are also within the scope of the invention. The conditions used to produce a new batch, lot, or a biosimilar of recombinant protein of interest may then be altered with the objective of altering the quantity or type of post-translational modifications of the recombinant protein to at least approximately match that of the reference product. A new batch of the recombinant protein is then prepared and a second in vitro pharmacological model is produced by repeating steps b) to d) outlined above. The second in vitro pharmacological model may then be compared with the in vitro pharmacological model for a reference product or with the original in vitro pharmacological model of said new batch, lot, or a biosimilar of a reference product. This procedure provides a means to assess the impact of the alterations of the post-translational modifications on the pharmacology of the recombinant protein.

The process of altering the production conditions and deteiinining additional in vitro pharmacological models may be repeated until a desired, altered or improved in vitro pharmacological model is obtained, approximately matching or surpassing the in vitro pharmacological model for a reference product, respectively. The recombinant protein production conditions used to obtain the in vitro pharmacological model matching that of a reference product may then be selected for batch production of a biosimilar of said recombinant protein while the altered or improved in vitro pharmacological model may then be selected for production of an optimized version of the recombinant protein which represents a “bio-better” version of the original pharmaceutical product.

In another aspect, there is provided a method for obtaining an in vitro pharmacological model of a recombinant protein in a host. The method includes the steps of:

a) selecting a plurality of biomolecules known or suspected to influence pharmacology of the recombinant protein in the host via a binding interaction with a post-translational modification of the recombinant protein;

b) contacting the recombinant protein with one of the biomolecules;

c) determining binding kinetics parameters of the recombinant protein to the biomolecule; and

d) repeating steps b) and c) with additional selected biomolecules to produce a set of binding kinetics parameters. This set of binding kinetics parameters represents an in vitro pharmacological model of said recombinant protein in the host which, in turn, provides a representation of in vivo pharmacological profile of the recombinant protein in the host.

In another aspect, there is provided a system for production and analysis of a new batch, lot, or a biosimilar of a reference product. The system includes a bioreactor for production of the recombinant protein and an assay module for determining binding kinetics parameters of the recombinant protein to a plurality of biomolecules. The assay module may be in liquid handling communication with the bioreactor for delivery of a recombinant protein to the assay module or can be directed manually. The system includes a library of individual biomolecules, which may be in liquid handling communication with the assay module for transfer of individual biomolecule samples into the assay module. Also included in the system is a controller for receiving output from the assay module and for input of instructions for delivery of an individual biomolecule solution to the assay module. The controller is further configured for providing a report which includes an in vitro pharmacological model for the recombinant protein.

The bioreactor may be provided with means for altering conditions for production of the recombinant protein. The means for altering conditions may be in digital data communication with the controller so that an operator may alter production conditions by providing input to the controller. Conditions which may be altered using the controller include, but are not limited to: temperature, pressure, gas flow, agitation, and composition of growth medium components. Examples of growth medium components include, but are not limited to biomolecules such as carbohydrates, salts, proteins and fats as well as commercially-obtained growth media or other components necessary for robust production of cells expressing the recombinant protein of interest.

The biomolecule library may include lectins, receptors, antibody-binding proteins or any other biomolecule which is known or suspected to interact with the recombinant protein in the host and to influence the pharmacological profile of the recombinant protein. Examples of such biomolecules include, but are not limited to, FcRn, Fc-gamma receptor I, Fc-gamma receptor II, Fc-gamma receptor III, collectin, mannose binding lectin, mannose receptor, ASGP-R, CL-K1, CL-P1, ficolin 1, ficolin 2, ficolin 3, siglec 1, siglec 2 and siglec 4. The biomolecule library may also include one or more additional biological drugs, which may be given as a combination therapy.

The system may be programmed to produce a report, which provides an assessment of the in vitro pharmacological model with respect to an in vitro pharmacological model of a reference product. The report is useful for determining how the alterations in conditions for production of the recombinant protein affect the in vitro pharmacological model and how they potentially affect the in vivo pharmacological profile of the recombinant protein in the host.

### BRIEF DESCRIPTION OF THE DRAWINGS

The foregoing summary and detailed description is better understood when read in conjunction with the accompanying drawings, which are included by way of example and not by way of limitation.

FIG. 1 is a process diagram indicating steps used in one embodiment of the present invention.

FIG. 2 is a schematic representation of a system for production and analysis of a recombinant protein in accordance with one embodiment of the present invention.

FIG. 3 is a schematic representation of a system for production and analysis of a recombinant protein in accordance with another embodiment of the present invention.

FIG. 4 provides a series of graphs comparing biomolecule binding to Herceptin relative to biomolecule binding to trastuzumab.

### DETAILED DESCRIPTION OF EMBODIMENTS

It is to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains.

In describing and claiming the present invention, the following terminology and grammatical variants will be used in accordance with the definitions set forth below.

To clarify the terms used herein are described hereinbelow:

The term “recombinant protein” refers to any protein species, produced in living cells, systems, or organisms. As used herein, the term “recombinant protein” includes but it is not limited to, proteins, polypeptides, and monoclonal or polyclonal antibodies.

As used herein the term “antibody” encompasses whole antibodies including single chain antibodies, and antigen whole antibodies, and antigen binding fragments thereof. Fab, Fab′ and F(ab′)2, Fd, single chain Fvs (scFv), single chain antibodies, disulfide-linked Fvs (sdFv) and fragments comprising either VL and VH are all within the present definition of the term “antibody.” Antibodies may originate from any animal origin including birds and mammals. Preferably, the antibodies are human, murine, rabbit, goat, guinea pig, camel, horse, or chicken.

The recombinant proteins can be post-translationally modified. Post-translational modifications on recombinant proteins include but are not limited to: glycosylation, carboxylation, hydroxylation, O-sulfation, amidation, glycylation, glycation, alkylation, acylation, acetylation, phosphorylation, biotinylation, formylation, lipidation, iodination, prenylation, oxidation, palmitoylation, pegylation, phosphatidylinositolation, phosphopantetheinylation, sialylation, and selenoylation.

Glycosylation, an attachment of oligosaccharides to the recombinant protein, is the most commonly found modification on recombinant proteins. O-Sulfation entails the attachment of a sulphate group to tyrosine, serine and threonine residues in a reaction catalyzed by sulfotransferases Amidation is characterized by the replacement of the C-terminal carboxyl group of a protein with an amide group. γ-carboxylation is characterized by conversion of glutamate residues to γ-carboxyglutamate (Glu→Gla). β-hydroxylation converts aspartate residues to β-hydroxyaspartate (Asp→Hya) or asparagine residues to β-hydroxyasparagine (Asn→Hyn).

The term “host,” as used herein, refers to a human or animal receiving a recombinant protein drug. Examples of a host include but are not limited to human, sheep, goat, cow, dog, cat, and horse.

The term “host proteins” refers to a class of biomolecules of the present invention. Host proteins are proteins of the host's biological system which interact with the recombinant protein of interest. Host proteins can be isolated from the host, produced using recombinant methods or expressed on cells such as CHO (Chinese Hamster Ovary). The host proteins are capable of interacting with a recombinant protein drug via peptide backbone or via a post-translational modification present on the recombinant protein drug. The FcRn receptor is an example of a host protein capable of binding to a monoclonal antibody via the protein backbone. Lectins are example of host proteins able to bind a recombinant protein drug via oligosaccharide post-translational modifications of proteins.

The term “biomolecules” include host proteins such as lectins, receptors, and antibody-binding proteins or any other biomolecules known or suspected to interact with the recombinant protein and influence the pharmacological profile of the recombinant protein within the host. Examples of such host proteins include, but are not limited to: FcRn, Fc-gamma receptor I, Fc-gamma receptor II, Fc-gamma receptor III, collectin, mannose binding lectin, mannose receptor, ASGP-R, CL-K1, CL-P1, ficolin 1, ficolin 2, ficolin 3, siglec 1, siglec 2 and siglec 4. The person skilled in the art will recognize that additional non-protein biomolecules may be known to interact with the recombinant protein of interest or may be subsequently discovered to interact with the recombinant protein of interest. The skilled person will recognize that methods using these other non-protein biomolecules are also within the scope of the invention.

The methods described herein are useful for modeling the pharmacological profile of a recombinant protein in vitro. The in vitro pharmacological model can be used to compare a new batch, lot, or a biosimilar of a recombinant protein drug to the reference product. The invention uses biological reactions, for example those that are able to recognize recombinant protein drug. In contrast with prior art methods the invention focuses on identification of the binding kinetics parameters between selected biomolecules and a recombinant protein. This enables the method of the invention to provide an in vitro pharmacological model, which represents the collection of binding kinetics parameter between selected biomolecules and the recombinant protein drug. The in vitro pharmacological model may be used to predict pharmacological properties of the recombinant protein drug candidate in the host and to optimize the production conditions of the new batch, lot or a copy of a recombinant protein drug.

The term “pharmacological profile” refers to a description of the effects of the host on a recombinant protein being used as a drug. The processes of binding of the recombinant protein may include binding of the recombinant protein by host proteins which include but are not limited to: lectins, antibody-binding proteins, receptors and other biomolecules known to those skilled in the art or additional biomolecules subsequently discovered to bind the biological drug. Tight binding (as represented by a dissociation constant as a binding parameter, for example) of the recombinant protein to one biomolecule may represent an unfavorable interaction which would lead to an unfavorable pharmacological profile while tight binding of the recombinant protein to another biomolecule may represent a favorable interaction. The complexity of biomolecular interactions dictates the pharmacological profile. A favorable pharmacological profile of a recombinant protein of interest may, for example, be the result of effective binding of the recombinant protein to a cell surface receptor, which facilitates its transport into the cell. In this example, it may be favorable if the binding to the receptor is not too tight because subsequent release of the recombinant protein into the cell may be delayed and prevent the recombinant protein from carrying out its desired function within the cell. The combination of several such interactions will dictate the overall pharmacological profile of a given recombinant protein drug.

The term “binding kinetics parameters” as used herein refers to the specific parameters describing an interaction between a recombinant protein and a biomolecule. Processes for determining binding kinetics parameters are well known to those skilled in the art of biochemistry. Herein, this term refers to the specific parameters describing interactions between host proteins and a recombinant protein. These parameters include but are not limited to maximum binding (Bₘₐₓ), dissociation constant (K_{d}), and a mathematical equation Y=BₘₐₓX/(K_{d}+X) describing the shape of the curve generated as a result of plotting the concentration of a recombinant protein drug on X axis versus the intensity of signal generated in the assay plotted on Y axis.

Bₘₐₓ refers to the maximum specific binding in the same units as Y axis. K_{d} is the equilibrium binding constant, in the same units as X axis and represents the concentration of a recombinant protein needed to achieve a half-maximum binding to a host protein at equilibrium. The skilled person will be cognizant that these parameters are outlined for the purposes of providing an example and that more complex biomolecular interactions between a given recombinant protein and host biomolecules (for example, the assembly of ternary or even quaternary binding complexes of mixtures of biomolecules) may necessitate the derivation of more complex kinetic equations with additional parameters yielding important information about the pharmacological profile of a recombinant protein of interest. These additional kinetic relationships and binding parameters derived therefrom are also within the scope of the invention. They include Kₒₙ and K_{off}.

| Variable | Name | Example Units |
|---|---|---|
| Kₒₙ | Association rate constant or on-rate constant | M⁻¹ min⁻¹ |
| K_{off} | Dissociation rate constant or off-rate constant | min⁻¹ |
| K_{d} | Equilibrium dissociation constant | M |

The term “in vitro pharmacological model” as used herein, refers to a collection of binding kinetics parameters determined using binding assays for a plurality of biomolecules which are known or suspected of interacting with a given recombinant protein of interest and influencing its pharmacological profile in a host. The in vitro pharmacological model is meant to provide an approximate representation of the actual in vivo pharmacological profile of the given recombinant protein of interest within a host. The in vitro pharmacological model is a useful and powerful tool because it can be prepared quickly and used to assess the effects of the conditions used to produce the recombinant protein. Optimization of production conditions is itself a complex and time-consuming effort because there are many factors which may influence the final product and therefore influence the pharmacological profile of the recombinant protein in a host. Without an in vitro pharmacological model it would be necessary to carry out in vivo investigations of the recombinant protein produced under various conditions in laboratory animals for example. This represents a significant investment in time and expense because in vivo testing is expensive and time consuming. The in vitro pharmacological model provides a means to avoid this extra time and expense in assessing production conditions.

The term “biosimilar” refers to a copy of an original biological drug (also known as “originator” drug or “branded” drug) approved by one or more drug approval agencies.

The term “bio-better” refers to a version to an original biological drug with the same protein sequence but different post-translational modifications, which affect the drug's biodistribution, pharmacokinetics and pharmacodynamics

As used herein, the term “candidate” with reference to biosimilar drug or bio-better drug, refers to the intent that it will be submitted for approval by one or more drug regulatory agencies of one or more different jurisdictions.

Host proteins interacting with a recombinant protein drug via peptide backbone or post-translational modifications within the scope of this invention include, but are not limited to, the host proteins listed hereinbelow.

FeRn is example of host protein known to interact with a protein backbone of antibodies, a subclass of recombinant proteins. FcRn is a heterodimeric protein consisting of a soluble light chain and a transmembrane anchored heavy chain (α-FcRn). FcRn functions as a “salvage receptor” regulating levels of circulating IgGs.

Lectins represent examples of host proteins capable of binding to oligosaccharide-modifications of recombinant proteins. Lectins belong to a diverse family of structurally unrelated proteins classified under one name because of their glycan binding properties, each with different sugar specificity and different localization. The extracellular subgroup of lectins which include C-type, R-type, siglecs, ficolins, 1-type and galectins are either secreted into the extracellular matrix or body fluids, or localized to the plasma membrane where they mediate a range of functions including cell adhesion, cell signaling, glycoprotein clearance and pathogen recognition.

FcRn is a heterodimeric protein consists of a soluble light chain and a transmembrane anchored heavy chain (α-FcRn). FcRn functions as a “salvage receptor” regulating levels of circulating IgGs.

Fc-gamma receptor I (also known as FcγRI and CD64), Fc-gamma receptor IIa and b (also known as CDw32 and FCγRIII and C16) are located on monocytes, NK cells, neutrophils. All except Fe-gamma RIIb mediate phagocytosis and enhance presentation of antibody coated antigens, leading to effective stimulation of both CD4+Th1 and CD8+CTL effector responses. Fc-gamma RIIIb abrogates all of these effects.

Collectins (collagen-like lectin) are members of a subgroup of C-type (i.e. Ca²⁺-dependent) animal lectins characterized by the presence of collagen-like sequences (Gly-Xaa-Yaa triplet).

Mannose binding lectin (MBL) or MBP (mannan-binding protein) belongs to a family of secreted collectins and binds to mannose or N-acetylglucosamine (GlcNAc) in a calcium-dependent manner Binding of MBL to pathogens triggers the activation of the lectin pathway.

The human macrophage mannose receptor (MMR, also known as CD206 and MRC1 (mannose receptor C, type 1)), is a 190 kDa scavenger receptor that is expressed on macrophages, myeloid dendritic cells, liver and lymphatic endothelial cells. Its extracellular region is composed of an N-terminal cysteine-rich domain, followed by a single fibronectin type II repeat, and eight C-type lectin carbohydrate recognition domains (CRD). The cysteine-rich domain mediates recognition of sulfated N-acetylgalactosamine which occurs on some extracellular matrix proteins and is the terminal sugar of the unusual oligosaccharides present on pituitary hormones such as lutropin and thyrotropin.

The human asialoglycoprotein receptor (ASGPR) is an endocytic recycling receptor that belongs to the subfamily of the C-type/Ca+2 dependent lectins.

Collectin kidney 1, (CL-K1, also known as collectin subfamily member 11 (COLEC11), is a 37 kDa collectin that circulates in the blood.

Human CL-P1 is known to be expressed in vascular endothelial cells. CL-P1 may play a role in bacterial recognition or as a scavenger receptor for desialylated proteins.

Ficolins are GlcNAc-binding lectins found in serum. All ficolins have a collagen-like domain and a fibrinogen-like domain. Ficolin-1 is found on the surface of circulating monocytes. The FBG domain of ficolin-1 binds microbial ligands that contain acetylated compounds. Ficolin-1 has been shown to bind N-acetyl glucosamine, N-acetyl galactosamine and sialyl-N-acetyl-lactosamine. Ficolin-2 (also L-ficolin or ficolin-B) is expressed in the liver and released into the circulation. Ficolin-2 binds microbial ligands that contain acetylated compounds including N-acetyl glucosamine in compounds such as lipoteichoic acid in gram-positive bacteria. Human ficolin-3 (fibrinogen/collagen-like) is expressed by bile duct epithelial cells and hepatocytes, and is released into the bile and circulation. It is also secreted by bronchial and alveolar epithelial cells in the lung. Ficolin-3 binds a limited set of carbohydrates containing mannose, galactose or D-fucose.

Siglecs are type I transmembrane proteins with an ability to bind sialic acid. Siglecs have a conserved arginine residue, which makes an essential electrostatic interaction with sialic acid. CD33-related siglecs have between 1 and 4 C-set domains and feature cytoplasmic tyrosine-based motifs involved in signaling and endocytosis. Each is expressed on a specific combination of cells of the immune system. B cells and monocytes each express a number of siglecs, but some members of the siglec family are also expressed on NK cells, neutrophils, basophils, eosinophils, dendritic cells and macrophages. The second subgroup of siglecs consists of siglecs 1, 2 and 4 (more frequently known as sialoadhesin, CD22 and myelin-associated glycoprotein/MAG) in both human and mouse. Sialoadhesin is expressed on macrophages. CD22 is found on B cells and, in addition to acting as an adhesion receptor, it has a number of tyrosine-based motifs in the cytoplasmic tail, which mediate signaling processes regulating B cell activity. CD22 selectively binds to glycans with Sia-2-6Gal-sequences including a biantennary N-linked glycans.

In one embodiment of the invention, the methods described hereinabove and illustrated in more detail hereinbelow may be used to develop an in vitro pharmacological model for a recombinant protein. The in vitro pharmacological model is useful for assessing a given batch of the recombinant protein or optimizing of a new batch of the recombinant protein produced under altered production conditions. Inasmuch, the in vitro pharmacological model permits the assessment of phaimacological properties of batches of recombinant proteins as drug candidates providing comparisons therebetween. For example, comparing the binding kinetics parameters for a reference product and its copy thereof allows assessment of similarity and thus enables development of a “biosimilar” version of the original drug. The invention is therefore also directed to the use of the methods described hereinabove in the development and optimization of “biosimilar” candidates. The “biosimilar” candidate may be further optimized by altering production conditions to improve post-translational modifications which may lead to a version of recombinant protein drug, which, on the basis of its in vitro pharmacological model, has a pharmacological profile similar to that of the originator drug.

The in vitro pharmacological model may be used for quality control of different batches of the recombinant protein drug. Comparison of the in vitro pharmacological models of a new batch of the recombinant protein to the reference product provides an assessment of similarity and thus can be used to monitor batch-to-batch manufacturing consistency. One embodiment is therefore also directed to the use of the methods described hereinabove in assessment and monitoring of batch- to-batch consistency during product manufacturing.

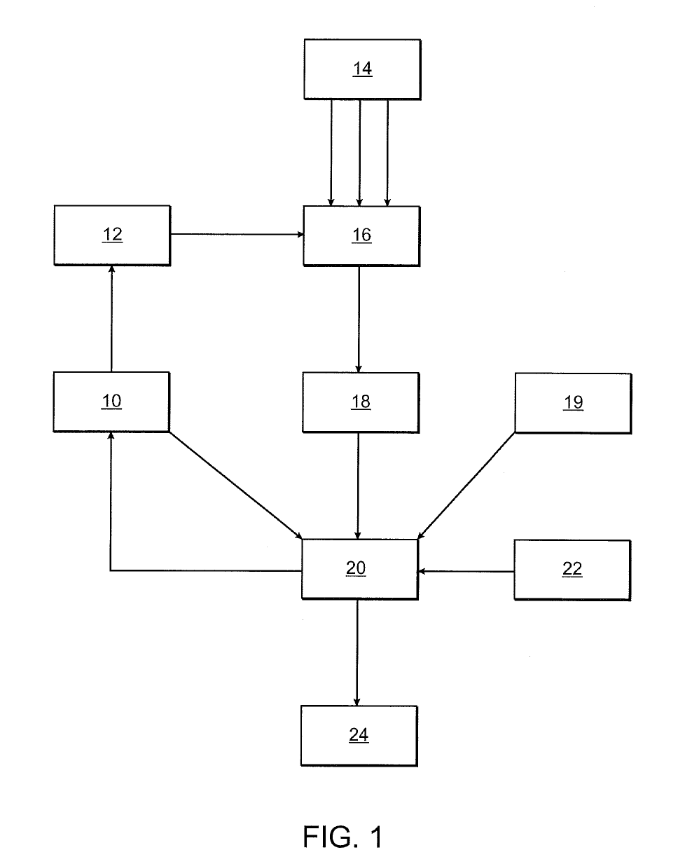

An exemplary embodiment of the method is outlined in FIG. 1. A set of production conditions 10 is used to produce a recombinant protein of interest 12. The recombinant protein 12 is contacted with a biomolecule from a selected group or library of biomolecules 14 which may include host proteins such as antibody-binding proteins, lectins, receptors or any other biomolecule known or suspected of influencing the pharmacological profile of the recombinant protein. Binding kinetics parameters 16 are measured to characterize the interactions of the recombinant protein of interest 12 with the biomolecule from the library of biomolecules 14. The aforementioned step is then repeated using additional biomolecules from the library of biomolecules as symbolized by the three arrows extending from 14 to 16. The experimentally-determined binding kinetics parameters 16 corresponding to the biomolecules investigated is then used to assemble an in vitro pharmacological model 18 which provides a representation of the expected in vivo pharmacology of the recombinant protein of interest 12 in the host. An optional scoring algorithm 19 may be applied to the binding kinetics parameters 16 or the pharmacological model 18 in the preparation of a report 20 which in effect provides an assessment of the pharmacological model 18 with respect to a comparator in vitro pharmacological model 22. In some embodiments, the comparator in vitro pharmacological model 22 is produced for an original approved biological drug while in other embodiments the comparator 22 is an earlier in vitro pharmacological model produced for the recombinant protein of interest 12 produced under different production conditions 10. Advantageously, in addition to a description of the in vitro pharmacological model itself the report may contain all of the information from the scoring algorithm 19, the identity of the comparator 22 and the production conditions 10 used to produce the recombinant protein of interest 12.

Armed with this information, the production conditions 10 may then be altered appropriately for production of a subsequent batch of the recombinant protein of interest 12 as symbolized by the arrow extending from 20 to 10. The process may be repeated until an optimized report 24 is produced which contains an optimized in vitro pharmacological model. The optimized report 24 advantageously contains information about the production conditions 10 used to produce the optimized version of the recombinant protein 12. The information contained in the optimized report 24 may be subsequently used to produce additional batches of an optimized version of the recombinant protein 12.

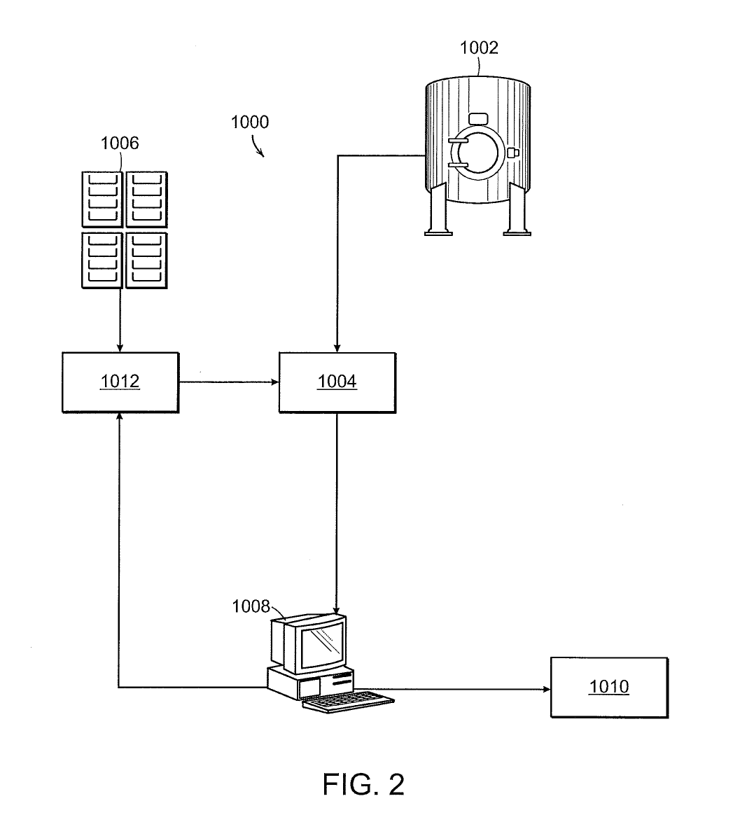

Another aspect of the invention is a system configured to utilize in vitro pharmacological modeling to drive the optimization of manufacturing conditions for said recombinant protein. One embodiment of such a system is shown in FIG. 2. In this embodiment, the system 1000 includes a bioreactor 1002 for growing cells used for production of the recombinant protein for which the pharmacological modeling analysis is being conducted. One or more liquid handlers (not shown) may be used for transferring liquids to and from various components if the system as described below. A sample containing the recombinant protein is drawn from the bioreactor 1002 manually or by a pump and transferred to assay module 1004. The assay module 1004 is configured to measure the binding kinetics parameters between the recombinant protein and a biomolecule such as a host protein, which is provided to the assay module 1004 from a biomolecule library 1006 via a biomolecule selector or manually by an operator 1012. The binding kinetics parameters generated by assay module 1004 are then transmitted or inputted by an operator to a computer 1008 which itself may be configured to operate the entire system or to operate discrete portions of the system in conjunction with other computers which may be linked in a local area network if so desired (not shown). The computer 1008 may also act as a controller for the liquid handlers of the system 1000. Configuration of liquid handlers for computer control is within the capabilities of one skilled in the art.

The computer 1008 may be provided with data analysis programs for determining binding kinetics parameters and to store these parameters for later analyses used in the development of a report 1010 containing an in vitro pharmacological model or a score-based representation thereof for the version of the recombinant protein, which was produced in the bioreactor 1002. The computer may also have a scoring algorithm programmed therein or stored on an appropriate computer-readable medium. The scoring algorithm may provide a weighted average to certain binding parameters or certain host proteins or combinations thereof, such that particularly important interactions of the recombinant protein with host biomolecules are weighted accordingly. The scoring algorithm may convert individual binding kinetics parameters or even an entire in vitro pharmacological model to a score which provides a measure of the parameters, or the model representing a desired pharmacological profile. The development of such scoring algorithms is within the capabilities of a person having ordinary skill in bioinformatics.

The computer 1008 may also be configured to control the biomolecule selector 1012 which selects a biomolecule from a biomolecule library 1006 and delivers the biomolecule to the assay module 1004 for measurement of the binding kinetics parameters against a newly drawn sample of the recombinant protein.

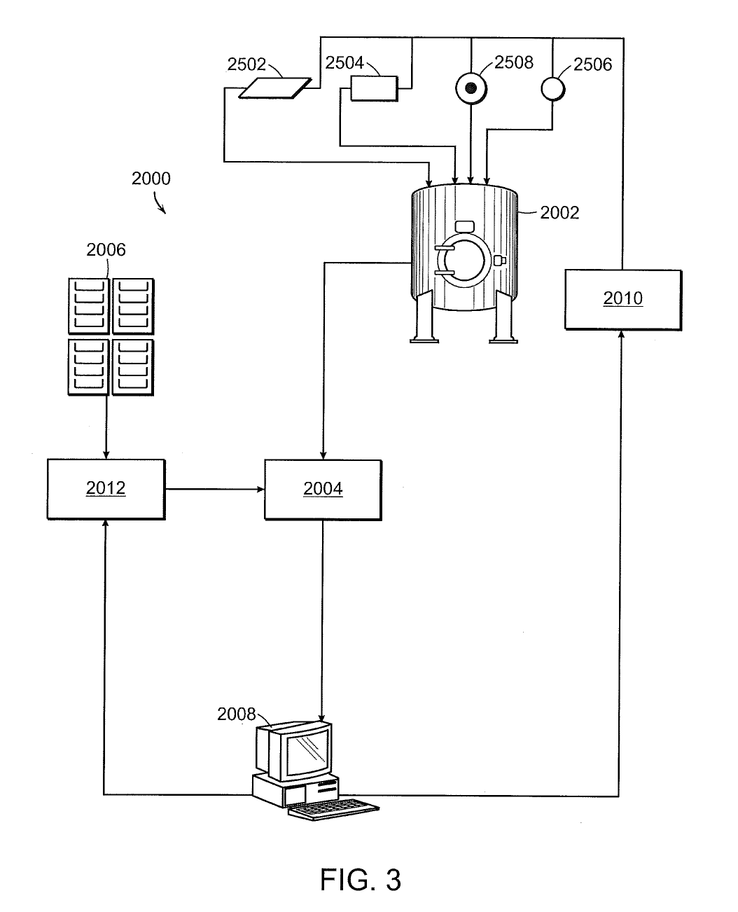

Another embodiment of the system is shown in FIG. 3. The system 2000 includes a bioreactor 2002 provided with a plurality of means for altering the production conditions of the recombinant protein of interest. Examples of such means for altering the production conditions shown in FIG. 3 include, but are not limited to, controllers for gas flow 2502, temperature 2504, pressure 2506 and the composition of the growth medium 2508 which is provided to the bioreactor 2002. The system 2000 includes an assay module 2004 for measurement of binding kinetics parameters with regard to binding of the recombinant protein of interest to a biomolecule such as a host protein, which is contained in a biomolecule library 2006. As described for FIG. 2, a computer 2008 may also be configured to control a biomolecule selector 2012 which selects a biomolecule from the biomolecule library 2006 and delivers the biomolecule to the assay module 2004 for measurement of the binding kinetics parameters against a newly drawn sample of the recombinant protein.

The raw data provided by the assay module 2004 are then processed manually or using the computer 2008 for construction of the in vitro pharmacological model. The scoring algorithm discussed above may also be employed if desired. The results are provided in a report 2010. The results of the report 2010 may be assessed and used as the basis for altering any or all of the conditions 2502, 2504, 2506 and 2508 in this embodiment. Preferably, the computer 2008 also controls the conditions of the bioreactor 2002 for production of a new version of the recombinant protein, although an additional computer or network workstation may be configured for this purpose. The new version of the recombinant protein may then be analyzed by repeating the steps outlined above. The system is thus configured for iterative production, analysis, and reporting of the results of the analysis which then feed into the logic for making choices for alterations in production conditions for the next iteration.

It is advantageous to validate the in vitro pharmacological models disclosed herein by comparing them to in vivo pharmacological data. This may be done by obtaining such pharmacological data using known in vivo analysis methods and comparing the in vivo data with the in vitro pharmacological models to ensure that the in vitro pharmacological models are providing adequate representation of the in vivo pharmacology of the recombinant protein being tested. The scoring algorithms outlined above may be developed using such validation data. To carry out such validation testing, samples of different batches of the recombinant protein being tested are saved for later in vivo testing. It is expected that once a given in vivo pharmacological model has been validated, such in vivo testing would no longer be necessary.

Assays for determining binding kinetics parameters used in preparation of the in vitro pharmacological model will be well known to those skilled in the art. In some embodiments, the assays include host proteins as the biomolecules. The host proteins may be prepared using recombinant methods to contain a tag. Tags used to modify biomolecules are well known to those skilled in the art. An example of a tag may include polyhistidine, glutathione S transferase, or myc.

In the method, a biomolecule from the specific host is retained at a constant concentration level while binding of the recombinant protein being tested is measured at varying concentrations. Commercially-available assay buffers may be used by a provider such as Perkin Elmer, for example. In some embodiments, donor beads tethered to a detection agent capable of specifically binding a tag on the host proteins are then added to the reaction at saturating concentration. Acceptor beads tethered to the detection agent capable of specifically binding a recombinant protein are added next to the reaction at saturating protein concentrations. A recombinant protein is then added to the reaction at different protein concentrations (preferably at least 6 different concentrations). When the donor and an acceptor beads are in close proximity to each other, the acceptor bead receives the energy from irradiated donor bead, which then emits light a specific wavelength to that acceptor bead. In homogeneous proximity assays, energy is transferred from a donor to an acceptor molecule when they are in close proximity to each other. The detection of light emission indicates the occurrence of binding between a host protein and the recombinant protein being tested. The assay described above is applied to multiple biomolecules, preferably in independent reactions, although it may also be possible to use more than one host protein in the same reaction. Characterization of binding interactions with several host proteins is expected to provide more accurate in vitro pharmacological models for the recombinant protein being analyzed.

Examples of homogenous proximity assays include, but are not limited to, time-resolved fluorescence resonance energy transfer (TR-FRET) assays. An example of a donor molecule is europium chelate and an example of an acceptor molecule is allophycocyanin (APC). The LANCE® technology uses Europium chelate (Eu) as donor dye which offers high quantum yield and a narrow-banded emission at approximately 340 nm. The acceptor dye, allophycocyanin (APC) receives energy from irradiated Eu chelate molecules in close proximity, and in turn emits light at 665 nm. APC is a fluorescent light harvesting protein unique to cyanobacteria and red algae and a member of the phycobiliprotein family of direct fluorescent dyes. Each of these assay components may be obtained from Perkin Elmer.

Another example of a homogenous proximity assay is AlphaScreen.® Donor and acceptor beads come in close proximity to each other when the recombinant protein drug and a biomolecule interact with each other. Laser excitation at 680 nm of a photosensitizer present on the donor bead induces the production of singlet oxygen. The singlet oxygen migrates to react with chemiluminescent moieties on the acceptor bead. The chemiluminescent moieties then activate fluorophores which emit light within the 520-620 nm range. Fluorescent labels require an excitation at one wavelength and detection at different wavelength. The methods for fluorescent detection are well known in the art. Methods of coupling fluorescent labels to proteins are also well known and adaptable to the current methods without undue experimentation.

Fluorescence readings obtained from the reactions between a biomolecule and a recombinant protein are then plotted with concentration of the recombinant protein on the X axis and the fluorescence reading representing the interaction or lack thereof on the Y axis. Binding kinetics parameters are determined from the plot. Bₘₐₓ is highest value of fluorescence where the graphed curve reaches a maximum value. K_{d} is the protein concentration at which 50% of the recombinant protein is bound to the host protein. This value is a recombinant protein concentration is equal to Bₘₐₓ/2. Another parameter utilized for this analysis is the exact shape of the binding curve which can be described using a mathematical equation.

In a further embodiment of the invention described above, surface plasmon resonance spectroscopy (SPR spectroscopy) can be used to determine binding kinetics parameters between the selected host proteins and a recombinant protein. SPR spectroscopy is an evanescent wave biosensor technology that monitors the interaction of two or more molecules in real-time. SPR biosensors are sensitive to changes in mass bound to the sensor surface and detect changes in refractive index. In this embodiment, there is no need to carry the reactions sequentially. Rather the invention provides a method whereby a multitude of reactions is carried out in parallel.

In yet another embodiment of the invention described above, selected biomolecules, most specifically host proteins, can be first expressed on mammalian cells allowing the biomolecules to be displayed on cell surface in their most native state. The examples of mammalian cells include but are not limited to CHO, A431 and other cell lines, each appropriate for the study of a particular recombinant protein and its reference product. Binding kinetics parameters of the recombinant protein and the reference product to these cells would then be evaluated using various methods including Enzyme-linked immunosorbent assay (ELISA) and SPR.

### EXAMPLES

### Example 1

### Measurement of Binding Kinetics Parameters using a Proximity Assay

This example illustrates measurement of binding kinetics parameters for a monoclonal antibody drug candidate for human use using a homogenous proximity assay. To obtain the binding kinetics parameters, a set of binding reactions between recombinant poly-histidine tagged biomolecules of human origin and a human monoclonal antibody drug are established. The set of human biomolecules may include any or all of the following proteins as well as additional human proteins known to interact with and influence the pharmacological profile of the specific monoclonal antibody drug candidate: FcRn, Fc-gamma receptor I, Fc-gamma receptor II, Fc-gamma receptor III, collectin, mannose binding lectin, mannose receptor, ASGP-R, CL-K1, CL-P1, ficolin 1, ficolin 2, ficolin 3, siglec 1, siglec 2 and siglec 4.

Each of the human biomolecules being analyzed is added to a binding buffer (Perkin Elmer) and plated at predetermined concentrations in a 96 well plate. Mouse anti-His antibody tethered to a Europium donor bead is added to each well at pre-determined concentrations. Rabbit anti-human antibody tethered to APC acceptor bead is added to each well at pre-determined concentrations. The recombinant human monoclonal antibody being tested is added to different wells at different protein concentrations for example, as follows: 10,000 nM, 1000 nM, 100 nM, 10 nM, 1 nM, 0.1 nM, and 0.01 nM in triplicate. Each reaction is allowed to reach equilibrium. The binding is then measured using a plate reader with excitation at 340 nm and emission at 665 nm. The fluorescence values obtained are plotted with the concentration of the recombinant monoclonal antibody drug on the X axis and fluorescence on the Y axis. A binding curve is then obtained for each set of binding reactions between a given human protein and a monoclonal antibody drug at different recombinant monoclonal antibody drug concentrations from which binding kinetics parameters are calculated. Binding kinetics parameters are obtained for each of the host proteins and assembled in a matrix form with K_{d}, Bₘₐₓ and a mathematical function which describes the shape of the binding curve. Processes for determining binding kinetics parameters are well known to those skilled in the art.

### Example 2

### Measurement of Binding Kinetics Parameters by SPR Spectroscopy

This example illustrates the use of SPR spectroscopy as a detection method for a binding assay for a recombinant monoclonal antibody. To obtain the binding kinetics parameters, the poly-histidine tagged human proteins listed in Example 1 are immobilized on the surface of a sensor chip. The monoclonal antibody is carried in a flow of buffer solution through a miniature flow cell. Binding of the antibody to an immobilized human protein on the surface of the sensor chip leads to a change in refractive index at the surface layer and is monitored by a detector such as a diode array. Time-dependent changes in the refractive index are recorded as sensorgrams. The sensorgrams provide information about binding or non-binding as well as providing information about the kinetics and the strength of the interaction.

### Example 3

### Construction of In Vitro Pharmacological Models for a Reference Product and its Use in Development of a “Biosimilar” Recombinant Drug Candidate

In this example, Protein Y is a reference product and Protein X is a copy of Protein Y. Binding assays are carried out for both Protein X (copy) and Protein Y (reference product) against a series of biomolecules including a selected group of proteins of human origin which are known to interact with and influence the pharmacology of both the reference product and its copy. For the purposes of illustrating this example, a series of five hypothetical human biomolecules is investigated. The skilled person will recognize that any number of host proteins or other classes of biomolecules which are known or suspected of interacting with and influencing the pharmacology of protein Y may be similarly investigated as long as the contribution of the additional biomolecules provides insight into biomolecular mechanisms and pharmacology of the recombinant protein.

Binding assays are well known to the skilled biochemist and can be developed without undue experimentation. Examples of binding assays which may be used are described in Examples 1 and 2. Other different binding assays may also be used. Binding kinetics parameters obtained from binding assays include but are not limited to dissociation constants, maximum binding and functions representing the shape of binding curves. For the purposes of this example, only the dissociation constant (K_{d}) and the maximum binding rate (Bₘₐₓ) will be employed as the parameters used in construction of the in vitro pharmacological model.

Firstly, a sample of the reference product (Protein Y) is obtained. If necessary, protein Y is isolated from its pharmaceutical formulation and subjected to a series of binding assays. The dissociation constant (K_{d}) and the maximum binding rate (Bₘₐₓ) are determined for each of the host proteins. The combination of these two variables across the series of host proteins makes up the in vitro pharmacological profile. The skilled person will recognize that while the present example is constructed with only five different host proteins, as more data become available for construction of pharmacological models and as more pharmacological models themselves become available for comparisons amongst themselves, more insight will be attained into the reliability of the in vitro pharmacological models as predictors of in vivo pharmacology.

TABLE 1
Comparison of In Vitro Pharmacological Models of Protein X
and Protein Y

| | Protein X | | Protein Y | |
|---|---|---|---|---|
| | (copy) | | (reference product) | |
| Biomolecule | | K_{d} | | K_{d} |
| Tested | Bₘₐₓ | (mM) | Bₘₐₓ | (mM) |
| Receptor A | 2.1 × 10⁻³ ± | 0.5 ± 0.05 | 0.52 ± 0.05 | 4.1 × 10⁻³ ± |
| | 0.05 × 10⁻³ | | | 0.05 × 10⁻³ |
| Lectin B | 6.4 × 10⁻³ ± | 2.1 ± 0.05 | 0.31 ± 0.05 | 5.3 × 10⁻³ ± |
| | 0.05 × 10⁻³ | | | 0.05 × 10⁻³ |
| Antibody Binding | 4.9 × 10⁻³ ± | 3.2 ± 0.05 | 0.58 ± 0.05 | 3.2 × 10⁻³ ± |
| Protein C | 0.05 × 10⁻³ | | | 0.05 × 10⁻³ |
| Receptor B | 5.0 × 10⁻³ ± | 1.5 ± 0.05 | 0.42 ± 0.05 | 1.1 × 10⁻³ ± |
| | 0.05 × 10⁻³ | | | 0.05 × 10⁻³ |
| Lectin D | 3.2 × 10⁻³ ± | 0.9 ± 0.05 | 0.76 ± 0.05 | 2.1 × 10⁻⁴ ± |
| | 0.05 × 10⁻³ | | | 0.05 × 10⁻⁴ |

To simplify the explanation of this example, the binding kinetics parameters for the reference product represent the desired binding kinetics parameters for the biosimilar (Table 1). Protein Y binds selected biomolecules at low K_{d} values and high Bₘₐₓ values. The skilled person will recognize that this assumption is simplistic and in reality, it may be advantageous to have weak interactions with certain biomolecules and strong interactions with others. Table 1 indicates that the values for K_{d} and Bₘₐₓ of Protein X across the series of biomolecules do not match the values for Protein Y. Therefore, the pharmacological model of Protein X is different from the reference product and needs to be further optimized. It is then considered that alteration of the conditions of production of protein X could promote favorable alterations in post-translational modifications of protein X which would provide enhanced pharmacology. The enhanced pharmacology would be recognizable in an updated in vitro pharmacological model of Protein X.

A new batch of Protein X (designated Protein X′) is then produced at different concentrations of growth media components such as glucose for example. It is expected that these conditions will alter the interactions with selected biomolecules and yield a Protein X′ of which in vitro pharmacological profile will be similar to that of Protein Y. Protein X′ is then harvested and the binding assays described above are repeated for Protein X′. The results are shown in Table 2.

TABLE 2
Comparison of In Vitro Pharmacological Models of Protein X′
and Protein Y

| | Protein X′ | | Protein Y | |
|---|---|---|---|---|
| | (copy) | | (reference product) | |
| Biomolecule | | K_{d} | | K_{d} |
| Tested | Bₘₐₓ | (mM) | Bₘₐₓ | (mM) |
| Receptor A | 2.7 × 10⁻² ± | 5.7 × 10⁻² ± | 0.52 ± 0.05 | 4.1 × 10⁻³ ± |
| | 0.05 × 10⁻³ | 0.05 × 10⁻³ | | 0.05 × 10⁻³ |
| Lectin B | 6.4 × 10⁻² ± | 9.4 × 10⁻² ± | 0.31 ± 0.05 | 5.3 × 10⁻³ ± |
| | 0.05 × 10⁻³ | 0.05 × 10⁻³ | | 0.05 × 10⁻³ |
| Antibody | 8.8 × 10⁻² ± | 2.7 × 10⁻² ± | 0.58 ± 0.05 | 3.2 × 10⁻³ ± |
| Binding | 0.05 × 10⁻³ | 0.05 × 10⁻³ | | 0.05 × 10⁻³ |
| Protein C | | | | |
| Receptor B | 2.9 × 10⁻² ± | 8.3 × 10⁻² ± | 0.42 ± 0.05 | 1.1 × 10⁻³ ± |
| | 0.05 × 10⁻³ | 0.05 × 10⁻³ | | 0.05 × 10⁻³ |
| Lectin D | 8.4 × 10⁻² ± | 6.1 × 10⁻³ ± | 0.76 ± 0.05 | 2.1 × 10⁻⁴ ± |
| | 0.05 × 10⁻³ | 0.05 × 10⁻³ | | 0.05 × 10⁻⁴ |

The results shown in Table 2 indicate that the modified production conditions have resulted in a recombinant protein X′ with an improved in vitro pharmacological model relative to Protein X. The in vitro pharmacological model of protein X′ more closely resembles that of Protein Y.

A third batch of the Protein X (designated Protein X″) is produced under conditions where additional parameters are modified, such as different temperature or a temperature shift, in addition to the alterations in growth medium components. It is now expected that the altered conditions may produce a recombinant protein (X″) with an in vitro pharmacological model more similar to the in vitro pharmacological model of protein Y, than Protein X′. The results of the next round of binding assays, shown in Table 3 indicate that the in vitro pharmacological model of Protein X″ has K_{d} and Bₘₐₓ, values that are similar to Protein Y.

TABLE 3
Comparison of In Vitro Pharmacological Models of Protein X″
and Protein Y

| | Protein X″ | | Protein Y | |
|---|---|---|---|---|
| | (copy) | | (reference product) | |
| Biomolecule | | K_{d} | | K_{d} |
| Tested | Bₘₐₓ | (mM) | Bₘₐₓ | (mM) |
| Receptor A | 0.58 ± 0.05 | 4.3 × 10⁻³ ± | 0.52 ± 0.05 | 4.1 × 10⁻³ ± |
| | | 0.05 × 10⁻³ | | 0.05 × 10⁻³ |
| Lectin B | 0.41 ± 0.05 | 4.9 × 10⁻³ ± | 0.31 ± 0.05 | 5.3 × 10⁻³ ± |
| | | 0.05 × 10⁻³ | | 0.05 × 10⁻³ |
| Antibody | 0.60 ± 0.05 | 3.5 × 10⁻³ ± | 0.58 ± 0.05 | 3.2 × 10⁻³ ± |
| Binding | | 0.05 × 10⁻³ | | 0.05 × 10⁻³ |
| Protein C | | | | |
| Receptor B | 0.49 ± 0.05 | 2.1 × 10⁻³ ± | 0.42 ± 0.05 | 1.1 × 10⁻³ ± |
| | | 0.05 × 10⁻³ | | 0.05 × 10⁻³ |
| Lectin D | 0.85 ± 0.05 | 2.3 × 10⁻⁴ ± | 0.76 ± 0.05 | 2.1 × 10⁻⁴ ± |
| | | 0.05 × 10⁻⁴ | | 0.05 × 10⁻⁴ |

On the basis of the comparison of the in vitro pharmacological model of Protein X″ to Protein Y, Protein X″ now represents a candidate for development of a biosimilar of Protein Y. The in vitro pharmacological model is then confirmed in model animals. It is evident that the in vitro pharmacological model has saved time and expense by refining the production conditions of the copied version of Protein Y, upfront, lowering the development time and cost associated with running extensive in vivo experiments.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention. Each reference (including, but not limited to, journal articles, U.S. and non-U.S. patents, patent application publications, international patent application publications, gene bank accession numbers, internet web sites, and the like) cited in the present application is incorporated herein by reference in its entirety.

### Example 4

### Measurement of Binding Kinetics Parameters using Fluorescence Activated Cell Sorting (FACS)

This example illustrates measurement and construction of in vitro Pharmacological Models for a reference product (Protein Y), such as Herceptin™ and its use in development of a biosimilar trastuzumab (Protein X) using biomolecules expressed on mammalian cells. An important determinant in success of establishing an in vitro Pharmacological Model is the cell line selection; selected cells should not bind the reference product before transfection. To establish an in vitro Pharmacological Model for a Herceptin™ reference product and its biosimilar, CHO cells were selected as they did not bind Herceptin™. For other reference products, other cell lines may be more appropriate and may be examined before establishing in vitro Pharmacological Model. Once cells are selected, a set of biomolocules is transiently or stably transfected into these cells. Various transfection protocols may be used to achieve this task. These protocols are well known to those skilled in the art. The biomolecules should be given sufficient time to be displayed on the outside of the transfected cells. To obtain the binding kinetics parameters and in vitro Pharmacological Model for the reference product and its biosimilar, a set of binding reactions between the transfected cells expressing the biomolecules and the drugs (the reference product and its biosimilar) for which the in vitro Pharmacological Model is being established. The set of biomolecules may include any or all of the following proteins, as well as additional human proteins known to interact with and influence the pharmacological profile of the specific monoclonal antibody drug candidate, human: FcRn, Fc-gamma receptor I, Fc-gamma receptor II, Fc-gamma receptor III, collectin, mannose binding lectin (MBL), mannose receptor, ASGP-R, CL-K1, CL-P1, Ficolin 1, Ficolin 2, Ficolin 3, Siglec 1, Siglec 2 and Siglec 4.

The binding of the reference drug and its biosimilar version to each biomolecule is measured using a FACS machine, for example. Each of the cell lines expressing a particular biomolecule is first added to a FACS binding buffer which is commonly a phosphate buffer containing a standard amount of bovine serum albumin Serum bovine albumin is used to reduce non-specific interaction between the cells and the biomolecules. The cells expressing various biomolecules are trypsinized and re-suspended at sufficiently high cell density in FACS binding buffer. The transfected cells displaying biomolecules are then plated in a 96 well plate in the presence of different concentrations of the reference product, in this case Herceptin™ (trastuzumab), or its biosimilar version produced under conditions X (Protein X). Both the reference product and the biosimilar being tested are then added to different wells containing cells displaying different biomolecules at different protein concentrations for example, as follows: 2,000 nM, 1000 nM, 500 nM, 250 nM, 125 nM, 62.5 nM. A reaction without a drug is used as a negative control and a baseline. Each reaction is allowed to reach equilibrium. The cells are then washed with FACS binding buffer and then fluorescently labeled with a secondary antibody specifically designed to specifically interact with only the reference product or the biosimilar and no other protein expressed on CHO cells. An example of a secondary antibody is a goat anti-human IgG-Alexa 647 (Invitrogen). The binding is then measured using a FACS machine using an appropriate fluorescence filter.

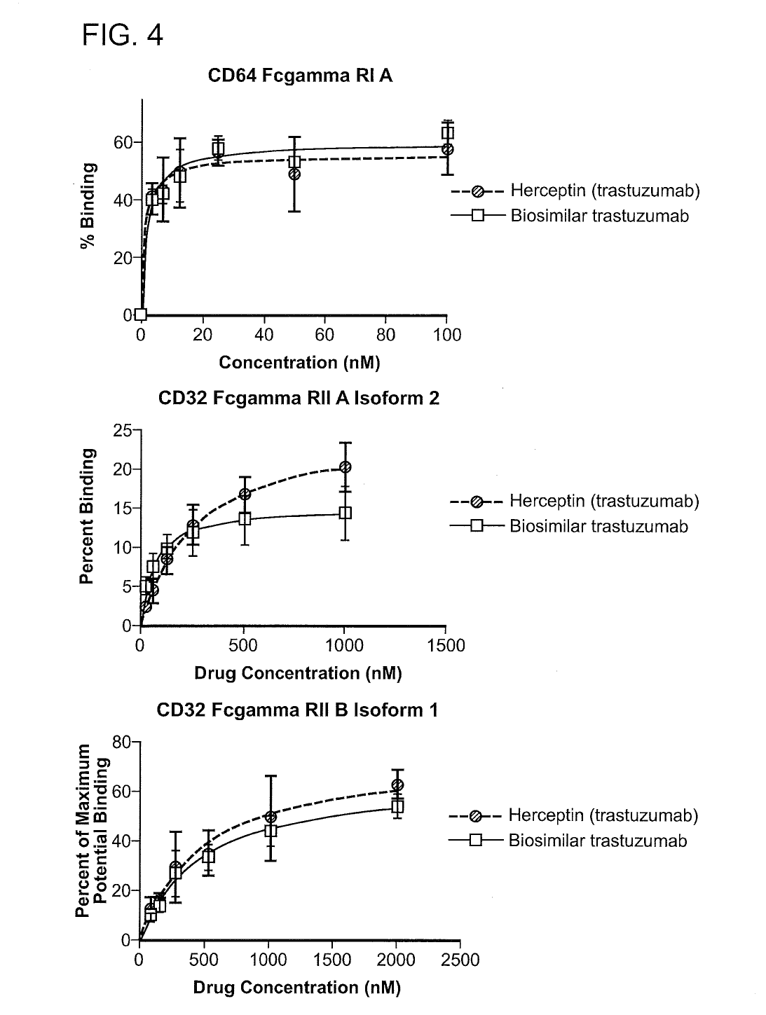

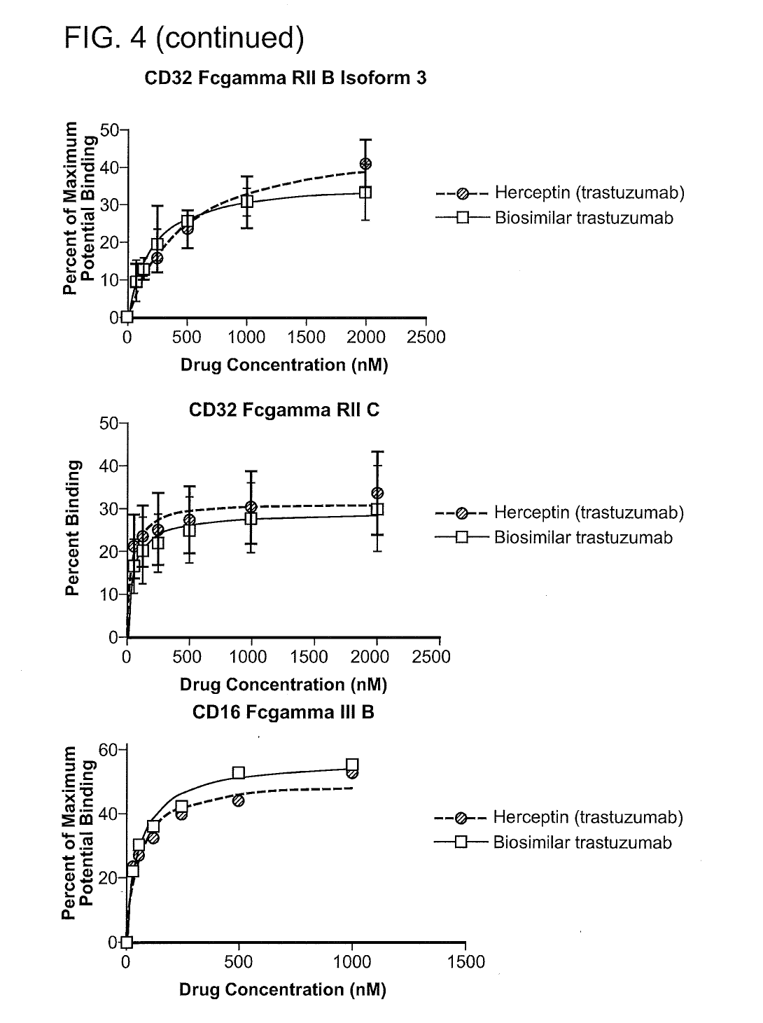

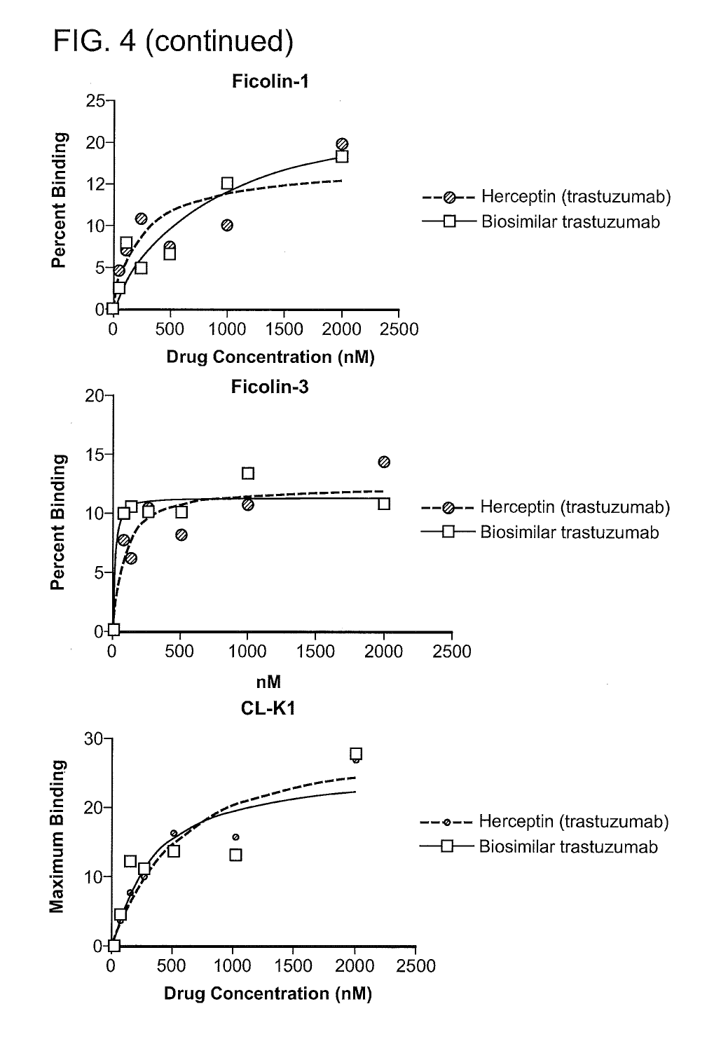

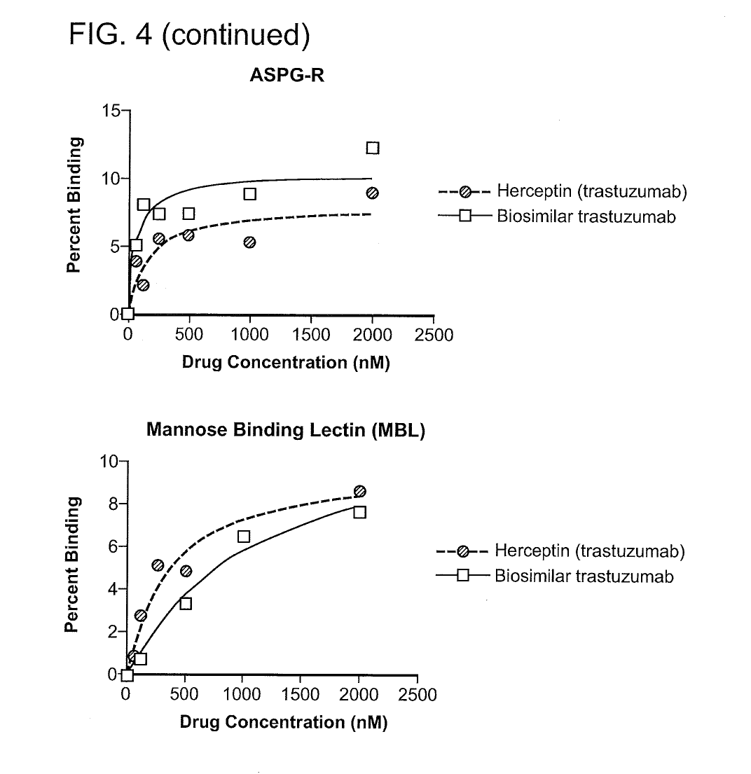

To obtain the in vitro pharmacological model the fluorescence values obtained are plotted with the concentration of the drug (reference product or its biosimilar) on the X axis and % of positively fluorescent cells on the Y axis. Another way to construct the in vitro pharmacological model to plot the concentration of the drug (reference product or its biosimilar) on the X axis and mean or median fluorescence shift on the Y axis. A binding curve is then obtained for each set of binding reactions between a cell line expressing a given biomolecule and the drug (reference standard or biosimilar) at different monoclonal antibody drug concentrations. Binding kinetics parameters are calculated from this curve using computer software with a capability of analyzing such binding data. Binding kinetics parameters are obtained for each of the biomolecules expressed in CHO and assembled in a matrix form with K_{d}, Bₘₐₓ (Table 4) derived from a function that describes the binding curve for both the reference product and its biosimilar shown on FIG. 4. The binding curves shown on FIG. 4 were prepared using Prism (GraphPad). Processes for determining binding kinetics parameters are well known to those skilled in the art.

TABLE 4
Comparison of In Vitro Pharmacological Models of Protein Y
(Herceptin ™ reference standard and Protein X (biosimilar Herceptin).

| | Pharmacologic profile | | | |
|---|---|---|---|---|
| | Bmax | Kd | Bmax | Kd |
| Biomolecule name | Herceptin ™ (trastuzumab) | | Biosimilar (trastuzumab) | |
| CD64 FcγRIA | 55.33 ± 2.19 | 1.3 ± .041 | 59.56 ± 2.72 | 2.0 ± 0.58 |
| CD32 FcγRIIA | 74.96 ± 2.55 | 277.5 ± 23.68 | 44.86 ± 0.34 | 71.10 ± 2.04 |
| Isoform 2 | | | | |
| CD32 FcγRIIB | 74.39 ± 6.125 | 457.6 ± 100.5 | 66.54 ± 3.05 | 407.3 ± 53.45 |
| Isoform 1 | | | | |
| CD32 FcγRIIB | 47.42 ± 4.2 | 440.2 ± 105.5 | 36.82 ± 0.82 | 201.9 ± 15.39 |
| Isoform 3 | | | | |
| CD32 FcγRIIC | 31.46 ± 1.47 | 29.16 ± 1.05 | 38.24 ± 11.57 | 59.92 ± 10.94 |
| CD16B FcγRIIIB | 50.63 ± 2.89 | 52 ± 12.5 | 56.96 ± 2.36 | 59.6 ± 9.96 |
| Ficolin-1 | 17.32 ± 4.14 | 259.7 ± 191 | 26.06 ± 7.58 | 856.3 ± 544 |
| Ficolin-3 | 12.19 ± 1.59 | 70.15 ± 44.66 | 11.32 ± 0.68 | 10.37 ± 9.86 |
| CL-K1 | 30.58 ± 4.69 | 511.9 ± 201.9 | 25.86 ± 6.16 | 321 ± 227.4 |
| MBL | 9.84 ± 1.25 | 347 ± 117 | 12.57 ± 2.48 | 1168 ± 475.1 |
| ASPGR | 8.063 ± 1.40 | 153.0 ± 98.46 | 10.38 ± 1.89 | 63.86 ± 33.89 |

Table 4 indicates that the values for K_{d} and Bₘₐₓ of Protein X (biosimilar trastuzumab) match that of the reference product (Protein Y) for the following biomolecules: CD64 FcγRIA, CD32 FcγRIIB Isoform 1, CD16B FcγRIIIB, Ficolin-1, CL-K1, ASPGR but do not match the series of the following biomolecules: CD32 FcγRIIA Isoform 2, CD32 FcγRIIB Isoform 3, Ficolin-3, CD32 Fc FcγRIIC, and MBL. If desired, the in vitro Pharmacological Model of biosimilar trastuzumab may be further optimized to match the in vitro Pharmacological Model for the reference product, Herceptin™ Further optimization is achieved by alterations in the conditions of production of biosimilar trastuzumab, Process X′.

A new batch of biosimilar trastuzumab (designated as Protein X′) is then produced at different cell growth conditions, which include alteration in concentrations of growth media components such as media feed and additives which include but are not limited to amino acids or glucose or galactose and glutamine. It is expected that these conditions will alter the interactions with selected biomolecules and yield a Protein X′ of which in vitro pharmacological profile will be similar to that of Protein Y. Protein X′ is then harvested and the binding assays described above are repeated for Protein X′, as described herein above.

What is claimed is:

1. A method comprising:
a) selecting a plurality of biomolecules known or suspected to influence a pharmacological profile of said recombinant protein in a host via one or more binding interactions with said recombinant protein;
b) contacting said recombinant protein with a first member of said plurality of biomolecules;
c) determining binding kinetics parameters of said recombinant protein to said first member using a binding assay; and
d) repeating steps b) and c) with additional members of said plurality of biomolecules to determine binding kinetics parameters for members of said plurality of biomolecules, thereby providing an in vitro pharmacological model of said recombinant protein in said host.

2. The method of claim 1 wherein said binding interactions are interactions of said biomolecules with the backbone of said recombinant protein or interactions with a post-translational modification of said recombinant protein.

3. The method of claim 2 wherein said post-translational modification is selected from the group consisting of: glycosylation, carboxylation, hydroxylation, O-sulfation, amidation, glycylation, glycation, alkylation, acylation, acetylation, phosphorylation, biotinylation, formylation, lipidation, iodination, prenylation, oxidation, palmitoylation, pegylation, phosphatidylinositolation, phosphopantetheinylation, sialylation, and selenoylation.

4. The method of claim 3 wherein said recombinant protein is a monoclonal antibody, a fusion protein or Fab fragment.

5. The method of claim 3 wherein said plurality of biomolecules comprises a host protein selected from the group consisting of lectins, receptors, and antibody-binding proteins.

6. The method of claim 5 wherein said host protein is selected from the group consisting of: FcRn, Fc-gamma receptor I, Fc-gamma receptor II, Fc-gamma receptor III, collectin, mannose binding lectin, mannose receptor, ASGP-R, CL-K1, CL-P1, ficolin 1, ficolin 2, ficolin 3, siglecs 1, siglecs 2 and siglecs 4.

7. The method of claim 3 further comprising:
e) comparing said in vitro pharmacological model for a new batch, lot or a biosimilar to the in vitro pharmacological model for a reference product obtained from performing steps a) to d).

8. The method of claim 7 further comprising:
f) altering recombinant protein production conditions used for producing said recombinant protein, wherein said production conditions influence the quantity or type of said one or more post-translational modifications of said recombinant protein;
g) preparing a new batch of said recombinant protein using said altered production conditions; and
h) repeating steps b) to d) thereby producing a second in vitro model of said pharmacological profile based on said new batch of said recombinant protein.

9. The method of claim 8 further comprising:
i) comparing said second in vitro pharmacological model with said in vitro pharmacological model for a reference product.

10. The method of claim 9 further comprising:
i) repeating steps b) to i) using additional alterations of said production conditions in each repetition until in vitro pharmacological model for a recombinant protein is similar to the in vitro pharmacological model of a reference product;
j) selecting said production conditions used in production of said similar in vitro pharmacological model for batch production of an optimized version of said recombinant protein.

11. The method of claim 10 wherein said optimized version is selected as an active ingredient for a biosimilar candidate or a bio-better candidate.

12. A method for obtaining an in vitro pharmacological model of a recombinant protein in a host, said method comprising:
a) selecting a plurality of biomolecules known or suspected to influence pharmacology of said recombinant protein in said host via a binding interaction with a post-translational modification of said recombinant protein;
b) contacting said recombinant protein with a first member of said plurality of biomolecules;
c) determining binding kinetics parameters of said recombinant protein to said first member using a binding assay; and
d) repeating steps b) and c) with additional members of said plurality of biomolecules to produce a plurality of binding kinetics parameters for members of said plurality of biomolecules, thereby providing an in vitro pharmacological model of said recombinant protein in said host.

13. The method of claim 12 wherein said post-translational modification is selected from the group consisting of: glycosylation, carboxylation, hydroxylation, O-sulfation, amidation, glycylation, glycation, alkylation, acylation, acetylation, phosphorylation, biotinylation, formylation, lipidation, iodination, prenylation, oxidation, palmitoylation, pegylation, phosphatidylinositolation, phosphopantetheinylation, sialylation, and selenoylation.

14. The method of claim 12 wherein said plurality of biomolecules comprises a host protein selected from the group consisting of lectins, receptors, and antibody-binding proteins.

15. A system for production and analysis of a recombinant protein, said system comprising:
a bioreactor for production of said recombinant protein,
an assay module for determining binding kinetics parameters of said recombinant protein binding to a plurality of biomolecules, said assay module in liquid handling communication with said bioreactor for delivery of a recombinant protein to said assay module;
a library of biomolecules, said library in liquid handling communication with said assay module for transfer of biomolecule samples into said assay module; and
a controller for receiving output from said assay module and for input of instructions for delivery of a biomolecule to said assay module, said controller further configured for providing a report comprising an in vitro pharmacological model for said recombinant protein.

16. The system of claim 15 wherein said bioreactor is provided with means for altering conditions for production of said recombinant protein, said means for altering conditions in digital data communication with said controller.

17. The system of claim 16 wherein said conditions are selected from the group consisting of: temperature, temperature shifts, pressure, gas flow, agitation, and composition of growth medium components.

18. The system of claim 15 wherein said biomolecule library comprises lectins, receptors and antibody-binding proteins.

19. The system of claim 18 wherein said biomolecule library comprises host proteins selected from the group consisting of: FcRn, Fc-gamma receptor I, Fc-gamma receptor II, Fc-gamma receptor III, collectin, mannose binding lectin, mannose receptor, ASGP-R, CL-K1, CL-P1, ficolin 1, ficolin 2, ficolin 3, siglecs 1, siglecs 2 and siglecs 4.

20. The system of claim 15 wherein said report provides an assessment of said in vitro pharmacological model with respect to an in vitro pharmacological model of a reference product.
